(19) United States
(12) Patent Application Publication
(10) Pub. No.: US 2001/0006780 A1
(43) Pub. Date: Jul. 5, 2001

(54) PROCESS FOR DETECTING A KNOWN SEQUENCE IN GENOMIC DNA

(76) Inventors: BENJAMIN W. BOLDT, ELM GROVE, WI (US); DENNIS A. ROSCETTI, WAUWATOSA, WI (US)

Correspondence Address: MARK K JOHNSON, P O BOX 510644, NEW BERLIN, WI, 531310644

(21) Appl. No.: 09/109,119

(22) Filed: Jun. 30, 1998

(51) Int. Cl. C12Q 1/68; C12P 19/34

(52) U.S. Cl. 435/6; 435/91.2

(57) ABSTRACT

Described is a process for screening genomic DNA samples for inherited conditions comprising selectively amplifying a portion of the genomic DNA using a primer that is substantially complementary to the portion but may have at least one mismatch nucleotide at or near one end. If the portion is amplified, hybridizing a probe to it, then, attaching the hybridized probe complex to a solid support and detecting the complex, if any.

### CROSS-REFERENCE TO RELATED APPLICATIONS

(Provisional Application Serial No.) (Filing Date) N/A

### FEDERALLY SPONSORED RESEARCH N/A

### FIELD

The present invention relates to a method and kit for detecting the presence or absence of one or more nucleotide sequences by amplification. The process is of particular interest in the diagnostic testing of DNA samples for genomic conditions whether inherited, such as mutations, deletions and polymorphisms, or occurrences to the genome not inherited, such as environmentally induced mutations, deletions, substitutions and additions, and provides a general method for detecting point mutations. It is also useful in the detection and typing of infectious pathogens by analysis of their DNA.

### BACKGROUND

Several hundred genetic diseases are known to exist in man, which result from particular mutations at the DNA level. The molecular basis for certain of these diseases is already known and research is rapidly revealing the molecular basis for those genetic diseases for which the nature of the mutation is at present unknown. Where the precise molecular basis for the inherited condition is not known, diagnosis of the disorder or location of carriers may be provided in informative pedigrees by restriction fragment length polymorphism (RFLP) technology using DNA probes in genetic linkage with the disease locus.

Thus, at present Duchenne Muscular Dystrophy, Cystic Fibrosis and Huntington's Chorea among others may be diagnosed using RFLP technology. However, such testing needs to be performed separately in respect to each condition and a substantial amount of work is required, each case likely requiring DNA purification, restriction enzyme digestion, agarose gel electrophoresis, Southern blotting, hybridization, detection of hybridized gene probe and pedigree analysis. Certain other inherited conditions are known to be associated with single point mutations or polymorphisms in genes, but each of these conditions must be analyzed separately and further particular difficulties arise where the point mutations are heterogeneous. This can involve complex RFLP haplotype analysis with multiple restriction enzymes.

Polymorphisms in HLA sequences are also known to be associated with disease conditions. Proteins encoded by polymorphic loci are most commonly typed using serological methods. One of the limitations of serological typing is that it does not differentiate between many of the alleles that are known to exist in the population. This has prompted the development of methods for analysis of HLA as well as other allelic polymorphisms at the genetic level.

The oldest method for typing, Southern Analysis, is based on immobilizing genomic DNA onto a solid phase, such as nitrocellulose or nylon membrane, and probing that material with a radiolabeled oligonucleotide “probe.” The nucleic acid sequence of the probe was selected to be complementary to a segment of the captured genomic material that included a known polymorphism. The ability to correctly interpret the test sample was dependent on the binding efficiency of the probe to the captured and denatured genomic DNA. In turn, the binding efficiency of the probe was dictated by the amount of time the probe was exposed to its potential target, as well as the composition and temperature of the hybridization buffer. In practice, in order to reduce non-specific binding of the probes, conditions are selected that slightly disfavor probe binding. Consequently, it is necessary to have a sufficient number of copies of target material and a very sensitive method of detection, as found with radiolabeled probes.

Coupling Southern analysis and restriction fragment length polymorphisms (RFLPs) made improvements to this strategy. The DNA was first digested by a restriction enzyme that cleaved at a specific sequence through out the genomic material. The resulting fragments were then size-fractionated by gel electrophoresis prior to transfer to the membrane. The bound material was then visualized with a probe that would bind to a relevant genomic segment. So, if the formation or deletion of a specific restriction enzyme site could describe a polymorphism, then, following probing of the digested and transferred material, the resulting fragment pattern would provide the bulk of information for interpretation of the sample. With this strategy, the probes became a mechanism for visualizing the results and were not used to identify specific polymorphisms.

The role of the probe was eliminated by the introduction of the polymerase chain reaction (PCR), as described in U.S. Pat. No. 4,683,202, issued Jul. 28, 1987. With the PCR, analysis of test samples could be focused entirely on the segments of genomic DNA containing the purported polymorphism. Mimicking the process of DNA replication, oligonucleotides bind to complementary regions and “prime” DNA strand synthesis by a DNA polymerase. Cis-positioned primers limit the size of the segment that is produced. The reaction is repeated many times to generate large quantities of a particular segment of genomic DNA. The amplified material, subjected to the restriction enzyme, was in sufficient quantity to allow the resulting fragments to be visualized directly in a gel following electrophoresis and staining with DNA-intercalating dyes.

Unfortunately, the elimination or formation of a restriction site does not describe all polymorphisms. Consequently, for some polymorphisms, discrimination is again dependent on the performance of sequence-specific (allele-specific) probes. Nevertheless, because the PCR produced large quantities of a specific segment for analysis, the amount of time required for hybridization was reduced. Moreover, because both the oligonucleotide probe and target amplification product are of a defined size, could be produced in large quantities, and the availability of non-isotopic detection systems, several configurations for DNA testing developed.

One strategy reflects the original method of DNA analysis: the amplification product was bound directly to a solid phase support (membrane, microtiter well, etc.), denatured by heat or chemicals, and probed. This procedure may be recognized as the “dot-blot” method. Alternatively, one could create a “reverse dot-blot” strategy by first attaching the probe to the solid phase and subsequently introducing the denatured amplification product. More recently, researchers have used sequence-specific oligonucleotide (“SSO”) probe hybridization to perform HLA-Class II typing. That method entails amplifying a polymorphic region of an HLA locus using the PCR, contacting the amplified DNA to a plurality of sequence-specific oligonucleotide probes under hybridizing conditions, and detecting hybrids formed between the amplified DNA and the sequence-specific oligonucleotide probes. A distinct advantage of this reverse dot-blot method is that it enables multiple amplification products, produced in a single PCR, to be conveniently introduced to multiple separate probes previously aliquotted. A single test sample can be analyzed for multiple alleles, simultaneously. Unfortunately, this multi-allelic analysis presupposes that all probes will work under similar conditions of time and temperature.

An alternative strategy again eliminates the role of discriminating probes by incorporating specificity into the amplification reaction by using sequence-specific primers. This approach capitalizes on the lack of a 5′ editing function in Taq polymerase; the DNA polymerase most often used in the PCR. The absence of this enzymatic function enables a nucleotide mismatch at or near the 3′ end of a primer to prevent extension of that primer, and the failure to form a PCR product. In this approach, primers are designed to terminate at or near the site of a known polymorphism with the ultimate base being distinctive for either the wild type or mutant base. The primers are said to be sequence-specific (SSP) or allele-specific (ASP), and the whole process is called amplification refractory mutation system (ARMS.) Following PCR, the presence or absence of an amplification product indicates the presence or absence of an allele in the genomic sample. However, it should be appreciated that the disruptive nature of a base mismatch does not ensure that an extension product will not be formed. Consequently, subjective interpretation of signal intensity may be required when analyzing results and assigning genotype to a test sample.

Since the absence of a product is indicative of an absent allele, it is necessary to co-amplify a control product to ensure PCR performance. However, in situ detection protocols do not differentiate PCR products. As a consequence, ARMS products are, traditionally, size-fractionated by gel electrophoresis and visualized in the gel. As described, the gel endpoint is amenable to identifying multiple ARMS products formed in a single reaction. In practice, constraining primer position and sequence to obtain a product of a specific size can negatively impact the yield of product, fragment visualization, and, subsequently, genotype assignment.

In summary, current technology used for identifying known DNA polymorphisms is limited. The PCR/reverse dot-blot format is capable of producing a large number of fragments in large quantity (due to the efficient binding of complementary primers in the PCR), but is limited by the need to use high stringency conditions for the hybridization of probes. ARMS format is limited by the number of samples that can be simultaneously amplified, because interpretation is based on identification of fragment size as well as presence. Both approaches require many rounds of amplification to produce enough material to either accommodate the inefficiency of probe binding to acquire probe specificity or to visualize the fragments directly, increasing the likelihood of identifying weakly amplified fragments as false-positives. There is a need for a simple method for directly detecting at least one single base difference in nucleic acids such as genomic DNA in which detection steps are minimized resulting in a method which may be performed quickly, accurately and easily with minimal operator skill. The present invention addresses some of the shortcomings of known typing methods and provides an improved process for resolving HLA and other alleles and for resolving allelic combinations.

### SUMMARY

By selecting the nucleotide sequence of an oligonucleotide primer appropriately it is possible to selectively achieve primer extension of either a sequence containing a suspected mismatch nucleotide or the corresponding sequence containing the normal nucleotide or to prevent such primer extension thus substantially simplifying the detection procedures necessary.

Provided is a method for detecting the presence or absence of at least one mismatch nucleotide in one or more nucleic acids contained in a sample by treating the sample with appropriate nucleoside triphosphates, an enzyme for polymerization of the nucleoside triphosphates and a detection primer for a diagnostic section of a target base sequence under hybridizing conditions, the nucleotide sequence of the detection primer being such that it is substantially complementary to the diagnostic section, a terminal nucleotide of the detection primer is either complementary to the suspected mismatch nucleotide or to the corresponding normal nucleotide, whereby an extension product of the detection primer is synthesized when the terminal nucleotide of the detection primer is complementary to the corresponding nucleotide in the target base sequence, no extension product is synthesized when the terminal nucleotide of the detection primer is not complementary to the corresponding nucleotide in the target base sequence; and detecting the presence or absence of the suspected mismatch nucleotide from the presence or absence of an extension product by attaching a sequence of nucleotides complementary to an extension product to a solid support for capturing an extension product.

While the method of the present invention is of particular interest in detecting the presence or absence of point mutations or polymorphisms in a preferred embodiment, the method is equally applicable to detecting the presence or absence of deletions, including deletions of more than one nucleotide as well as to detecting the presence or absence of substitutions of more than one nucleotide. In fact, the method is useful for detecting a difference of at least one base mismatch from a base sequence chosen by a person performing the test. For example, using the method described, one may choose to detect a genomic sequence that is different by at least one base from a known wild type sequence. The difference could be a deletion of one or more nucleotide bases, substituted bases, or even bases added to the genomic sequence to be detected. The difference may be attributable to an inherited mutation, deletion, substitution, addition, or polymorphism or it may be attributable to incidents to the genome other than genetic inheritance causing a mutation of one or more bases, deletion(s), substitution(s) or addition(s) to a known genomic sequence.

In a preferred embodiment of the present invention the method comprises treating the sample, together or sequentially, with appropriate nucleoside triphosphates, an enzyme for polymerization of the nucleoside triphosphates a detection primer for a diagnostic section of a target base sequence and a corresponding amplification primer under hybridizing conditions. The nucleotide sequence of the detection primer is substantially complementary to the diagnostic section. A terminal nucleotide of the detection primer is either complementary to the suspected mismatch nucleotide or to the corresponding normal nucleotide, whereby an extension product of the detection primer is synthesized when the terminal nucleotide of the detection primer is complementary to the corresponding nucleotide in the target base sequence.

No extension product is detectable in the present invention when the terminal nucleotide and/or one or more preceding nucleotides of the detection primer is not complementary to the corresponding nucleotide in the target base sequence; any extension product of the detection primer formed is capable of serving as a template for synthesis of an extension product of the amplification primer after separation from its complement.

The sample is treated under denaturing conditions to separate the primer extension product from its template where such extension product is formed. Another primer is then placed in contact with the single strands to synthesize further extension products using the single strands produced as templates. Repeat steps a sufficient number of times to result in detectable amplification of the appropriate nucleotide sequence. Finally, detect the presence or absence of the suspected mismatch nucleotide from the presence or absence of an amplification product obtained.

If desired the detection primer may carry a signal or label which would not be at risk of destruction, for example in a high temperature cycling technique such as PCR. For example labeling may be effected using an appropriate labeling or signaling moiety, such as alkaline phosphatase or horseradish peroxidase.

Further, using the process as described, the presence or absence of a PCR product is indicative of the presence or absence of an allele. Then, capturing it with a specific oligonucleotide assesses the presence or absence of that product. Since size fractionation (required in the ARMS type interpretation) is not required here, multiple fragments can be generated without regard to the size of the final products. These fragments can then be identified by capturing them with an oligonucleotide that is specific for the segment(s) of genomic material being amplified in sequence-specific manner. In addition, because the individual capture probes are different from one another, reflecting the distinct regions of the genome being amplified, the hybridization conditions can be less stringent to allow more efficient binding in a shorter time period. Furthermore, because the binding of the probe is efficient, fewer targets needs to be generated; which means that fewer rounds of amplification are required, decreasing the chance for the amplification of false positives. Nevertheless, if a given SSP reaction is routinely producing false positive results or to minimize the likelihood of observing contamination, the stringency of the hybridization reaction can be increased or the probe sequence modified to reduce binding efficiency. Taken further, it is possible to design probes to introduce additional allele specificity if necessary to genotype highly polymorphic loci of genomic DNA, such as HLA.

Described is a process for testing genomic DNA for genomic conditions whether inherited, such as mutations, deletions, additions and polymorphisms, or occurrences to the genome not inherited, such as environmentally induced mutations, deletions, substitutions and additions, comprising: forming a solution comprising the genomic DNA; adding a primer substantially complementary to a diagnostic section of the genomic DNA, selected from the group consisting of a primer having no mismatch bases and a primer having at least one mismatch base; mixing a DNA polymerase into the solution; amplifying the diagnostic section; capturing amplified polynucleotide strands to a solid support; and, detecting captured amplified polynucleotide strands.

A process for detecting a mismatch base in a diagnostic section of genomic DNA for conditions, whether inherited or not inherited, comprising: obtaining the genomic DNA; mixing the genomic DNA with a primer substantially complementary to the diagnostic section of the genomic DNA, selected from the group consisting of a primer having no mismatch bases and a primer having at least one mismatch base; selectively amplifying the diagnostic section from the genomic DNA; capturing amplified polynucleotides to a solid support; and, quantifying any complex attached to the solid support.

The present invention provides for kits having one or more receptacles for materials required including instructions for use. By the term “instructions for use,” it is meant a tangible expression describing the reagent concentration for at least one assay method, parameters such as the relative amount of reagent and sample to be admixed, maintenance time periods for reagent/sample admixtures, temperature, buffer conditions and the like.

A kit for testing genomic DNA for conditions, whether inherited or not inherited, comprising: a receptacle containing a primer having a nucleotide sequence substantially complementary to a diagnostic section of the DNA; a solid support; and, a receptacle containing a reporter label.

Further objects, features, and advantages of the invention will be apparent from the following detailed description when taken in conjunction with the accompanying drawings.

### BRIEF DESCRIPTION OF THE DRAWINGS

FIG. 1 is a graphical representation of results obtained genotyping 20 DR52a positive and 20 DR52a negative samples illustrating 100% correlation between the original sequencing of the samples and the process described herein.

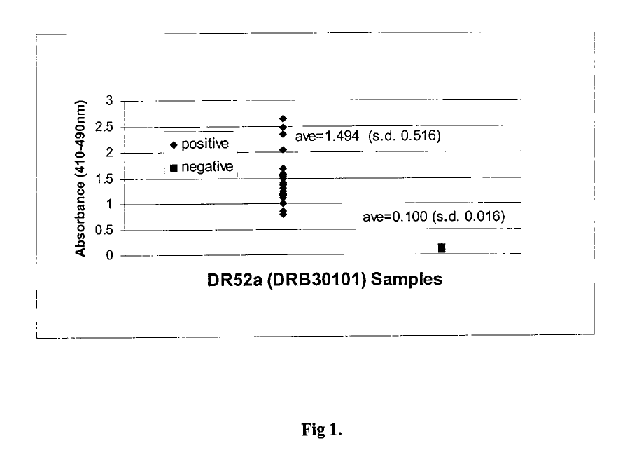

### DETAILED DESCRIPTION

The term “nucleoside triphosphate” is used to refer to nucleosides present in either DNA or RNA and thus includes nucleosides which incorporate adenine, cytosine, guanine, thyrine and uracil as base, the sugar moiety being deoxyribose or ribose. In general deoxyribonucleosides will be employed in combination with a DNA polymerase. However, other modified bases capable of base pairing with one of the conventional bases adenine, cytosine, guanine, thymine and uracil may be employed. If desired one or more of the nucleoside triphosphates present in the reaction mixture for the purpose of incorporation in to the extended primer(s) may be labeled or marked in any convenient manner.

The term “nucleotide” as used can refer to nucleotides present in either DNA or RNA and thus includes nucleotides which incorporate adenine, cytosine, guanine, thymine and uracil as base, the sugar moiety being deoxyribose or ribose. It will be appreciated however that other modified bases capable of base pairing with one of the conventional bases, adenine, cytosine, guanine, thymine and uracil, may be used in the detection primer and amplification primer employed in the present invention.

The enzyme for polymerization of the nucleoside triphosphates may be any compound or system which will function to accomplish the synthesis of primer extension products, including enzymes. Suitable enzymes for this purpose include, for example, E. coli DNA Polymerase I, Klenow fragment of E. coli DNA polymerase I, T4 DNA polymerase, other available DNA polymerases, reverse transcriptase, and other enzymes, including thermostable enzymes such as Taq polymerase. The term “thermostable enzyme” refers to an enzyme which is stable to heat and is heat resistant and catalyzes (facilitates) combination of the nucleotides in the proper manner to form the primer extension products which are complementary to each nucleic acid strand. Generally, the synthesis will be initiated at the 5′ end of each primer and will proceed in the 3′ direction along the template strand, until synthesis terminates, producing molecules of different lengths. There may be thermostable enzymes for example which initiate synthesis at the 3′ end and proceed in the other direction, using the same process as described above.

The expression “diagnostic section” means that portion of the genomic target sequence which contains at least one potential mismatch nucleotide, whether a deletion, addition, substitution or polymorphism; the presence or absence of which is being detected by the described process. Generally one of possibly a plurality of potential mismatch nucleotides will be a pairing base on the genomic strand opposite the 3′-terminal end of the primer extension sequence since, in a preferred embodiment, primer extension products will be initiated at the 5′ end of each primer as described above. The 3′-terminal end may include one or more of the five 3′ bases in the primer. Where, however, an enzyme for polymerization is to be used which initiates synthesis at the 3′ end of the detection primer and proceeds in the 5′ direction along the template strand until synthesis terminates the diagnostic section will contain the potential mismatch nucleotide near or at its 5′ end.

The term “oligonucleotide” as used herein is defined as a molecule comprised of two or more deoxyribonucleotides or ribonucleotides, preferably more than three. Its exact size will depend on many factors and the exact sequence of the oligonucleotide may also depend on a number of factors as described. The oligonucleotide may be derived synthetically or by cloning.

The term “primer” as used herein refers to an oligonucleotide, whether occurring naturally or produced synthetically, which is capable of acting as a point of initiation of synthesis when placed under conditions in which synthesis of a primer extension product which is complementary to a nucleic acid strand is induced, i.e., in the presence of appropriate nucleoside triphosphates and an enzyme for polymerization such as DNA polymerase in an appropriate buffer (“buffer” includes pH, ionic strength, cofactors, etc.) and at a suitable temperature.

The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the enzyme for polymerization. The exact lengths of the primers will depend on many factors, including temperature and source of primer and use of the method. For example, depending on the complexity of the target sequence, the detection and amplification primers typically contain 12-35 nucleotides, although they may contain more or fewer nucleotides. Short primer molecules generally require lower temperatures to form sufficiently stable hybrid complexes with the template.

The term “complementary to” is used herein in relation to nucleotides to mean a nucleotide which will base pair with another specific nucleotide. Thus adenosine triphosphate is complementary to uridine triphosphate or thymidine triphosphate and guanosine triphosphate is complementary to cytidine triphosphate. It is appreciated that while thymidine triphosphate and guanosine triphosphate may base pair under certain circumstances they are not regarded as complementary for the purposes of this specification. It will also be appreciated that while cytosine triphosphate and adenosine triphosphate may base pair under certain circumstances they are not regarded as complementary for the purposes of this specification. The same applies to cytosine triphosphate and uracil triphosphate.

The primers herein are selected to be substantially complementary to the different strands of each specific sequence to be amplified. This means that the primers must be sufficiently complementary to hybridize with their respective strands. Therefore, the primer sequence need not reflect the exact sequence of the template. For example, where the primer comprises a nucleotide sequence in which the 3′-terminal nucleotide is complementary to either the suspected mismatch nucleotide or the corresponding normal nucleotide a non-complementary nucleotide fragment may be attached to the 5′-end of the primer, with the remainder of the primer sequence being complementary to the diagnostic section of the target base sequence. Commonly the primers are complementary except when non-complementary nucleotides may be present at a predetermined primer terminus as described.

In certain circumstances synthesis of a detection primer extension product might be induced to occur even in the presence of a non-complementary 3′-terminal residue. This result may arise from the use of too low a temperature in which case the temperature may be increased, too long a time of incubation/annealing in which case the time may be reduced, too high a salt concentration in which case the salt concentration may be reduced, too high an enzyme concentration, too high a nucleoside triphosphate concentration, an incorrect pH or an incorrect length of oligonucleotide primer. A major source of incorrect extension products is probably allowing the reaction temperature to fall too low, thus permitting too low a stringency.

In addition to the above it may be found that incorrect results may also arise from use of a detection primer which is particularly rich in G (guanosine) and C(cytidine) residues. A detection primer may give rise to difficulty in this regard if it is G/C rich as a whole or particularly if it is G/C rich at its relevant, normally 3′, end. Moreover the precise nature of the base pairing in the region of the relevant, normally 3′, end of the detection primer when in use may be the cause of an incorrect result. Therefore, the presence of A's (adenosine) in the base pairing in the region of the relevant, normally 3′, end of the detection primer tends to improve specificity while the presence of G's (guanosine) does not. Furthermore the precise nature of the mismatch at the relevant, normally 3′, end of the detection primer may be an important factor in whether or not an inaccurate result is obtained. Thus for example an AA or CT mismatch does not normally result in hybridization, but a GT or AC mismatch may result in a sufficient degree of hybridization to result in the formation of inappropriate product(s). Inaccurate results may be avoided by deliberately introducing one or more further mismatched residues, or if desired, deletions or insertions, within the detection primer to destabilize the primer by further reducing the binding during hybridization.

In one embodiment, the method provides an improvement for HLA typing. The method involves selectively amplifying the HLA alleles and locating polymorphic sequences by sequence-specific oligonucleotide probe hybridization followed by solid support hybridization and detection.

The Human Leukocyte Antigen (HLA) typing methods according to the invention are useful as part of a method for tissue matching for transplantation. The information obtained from the typing can also be used for identification (forensic or paternity testing) and disease susceptibility such as for purposes of preventative therapy or insurance. For example, a potential bone marrow recipient and a pool of potential donors are HLA typed using standard serological and/or oligotyping procedures. If the recipient and any of the potential donors test positive for HLA oligotypes, or certain heterozygote combinations, HLA samples from the recipient and potential donors who test positive are further analyzed and the HLA oligotypes are further resolved according to the method of the invention.

Because HLA type is inherited from each parent, an individual may have up to eight different HLA alleles. A mixture will type the same as another mixture using conventional oligotyping, since each pair taken together contains the same combination of five polymorphisms, and oligotyping cannot reveal linkages between polymorphisms at widely spaced sites using a single probe.

However, sequence specific amplification can be used to resolve which of the alleles is present in such a situation. To determine which of the HLA alleles is present, the sample DNA is amplified with primers that selectively amplify specific HLA alleles. The amplified DNA is then hybridized to labeled oligonucleotides, and typing can be conducted with the knowledge that certain types are not present, since they were not amplified. In this manner, the two complementary pairs of alleles may be resolved.

The amplification product, double-stranded polynucleotides, are then chemically or heat denatured and placed on a solid support to hybridize (bind selectively) to sequence-specific capture probes previously attached to the solid support. A solid support may include, for example, polystyrene microtiter plates surface grafted with 0.2M glycidyl methacrylate and 2M benzophenone, in acetone by UV irradiating for 2 minutes. (K. Almer et.al, Polymer Chamistry 26, 1988, 2099-21110). Alternatively, 25 ug Streptavidin (Amersham, UK) in a TE buffer, at a total volume of 10 uL was applied to each well and the microtiter plate was incubated over night at 42.degree. C. After removing the supernatant, the wells were incubated with BSA, 100 ug/ml in TE-buffer, overnight at a total volume of 10 ul and 42.degree. C. The supernatant was removed and the wells subsequently washed with 1.×TE. The capturing oligonucleotides are modified to display biotin, the immunological partner to streptavidin. In addition, it is also possible to immobilize the capture probe by passive adsorption, by UV light, and by covalent binding of base modified DNA molecules by phosphoramidite linkage to a 5′ terminal phosphate or a peptide linkage to a terminal amine.

The sequence-specific probes are complementary to a diagnostic section of one strand of the double-stranded polynucleotides such that the probe anneals to the strand when in contact. PCR products that are not captured by sequence-specific probes are removed from the solid support by a series of washes. The captured sequence is termed a probe-polynucleotide complex in a preferred embodiment. To detect the bound sequences, add an oligonucleotide sequence termed a reporter label that is complementary to and binds to the captured polynucleotide. Alternatively, the reporter label may be any compound that specifically binds the captured polynucleotide for the purpose of providing a quantitation mechanism; for example, an immunological partner such as an antibody specific for a base, base sequence, or haptenated or otherwise altered base. In a preferred embodiment, the reporter label comprises a compound that produces a color when in contact with certain chemicals. Again, a series of washes removes unbound reporter labels from the microwell. A substrate is added that, when acted upon by the reporter label, produces a color, indicating a captured sequence and a positive result. Negative results produce substantially less or no color. The microwell can be visually inspected but preferably is read on a microplate reader to quantitate reactivity. A variety of methods can be used for determining the presence of a product, including, but not limited to, enzyme labels with appropriate substrate or analog, fluorescence, fluorescence polarization, luminescent labels, dyes, vesicle labels, and particle labels.

Examples of the present invention are provided for illustrative purposes and not to limit the scope of the invention.

### EXAMPLE 1

### Human Leukocyte Antigen (HLA) class II allele: DR52a

Sample Preparation

Using aseptic techniques, draw several mLs of venous blood into an EDTA tube and mix thoroughly by gently inverting the tube several times. Blood may be stored at 2-8° C. for several days before processing. Extract the DNA using either published techniques or a commercially available kit. Note that the method used for DNA isolation may dictate the volume and storage conditions of the blood. Resuspend the DNA in distilled water or 10 mM Tris (pH 7.0-7.3) to a concentration of 250 to 500 ng/μL. DNA that cannot be used immediately may be held at 2-8° C. for several days. For longer-term storage, samples should be stored frozen in a constant-temperature freezer. Excess contaminating protein, heparin, or EDTA may interfere with PCR amplification of the purified DNA.

### EXAMPLE 2

### Compositions

Master Amplification Mix contains next three (3) lines of materials

10 mM Tris (pH 8.3), 50 mM KCl, 0.01% Gelatin: Sigma [St Louis, Mo.]

200 μM dNTP(each): Pharmacia Biotech [Piscataway, N.J.]

0.5 μM primers: Custom synthesis from Genosys [The Woodlands, Tex.]

Primer 1 sequence=5′ FITC-modified-CTG TTC CAG GAC TCG GCG A 3′

Primer 2 sequence=5′ GCA CGT TTC TTG GAG CTG CG 3′

25-50 ng/μL genomic DNA

0.5 units/μL Taq polymerase: Perkin Elmer [Foster City, Calif.], Promega (Madison, Wis.)

Keep all necessary reagents refrigerated or on ice. Prepare all work surfaces and pipettors before use by wiping with 10% household bleach. Indicated volumes are based on an assay of six (6) samples with two (2) controls. For more or fewer samples, adjust number of wells and materials accordingly. Prepare a thin-walled amplification reaction tube for each sample, one positive control and one negative (environmental) control. Place tubes on ice.

Prepare 90 μL of a fresh working dilution of Taq DNA polymerase (final concentration of 0.2 U/μL) with molecular biology grade water in a microfuge tube placed on ice. If not amplifying 8 samples, then adjust the appropriate numbers in the following equations to determine the required volumes.

| | | |
|---|---|---|
| Number of samples | 6 | |
| Number of controls | 2 | |
| Add one for pipetting errors | 1 | |
| Total Number of Reactions | 9 | |
| Number of reactions | 9 | |
| Amount of Taq per reaction | × 2 | units |
| Total Units of Taq Required | 18 | units |
| Total units of Taq required | 18 | |
| Concentration of Taq stock | ÷ 5 | U/μL |
| Total Volume of Taq stock Required | 3.6 | μL. |
| Total volume required | 90 | μL |
| Volume of Taq | − 3.6 | μL |
| Volume of Water Required | 86.4 | μL |
| Total number of reactions | 9 | |
| Volume of Amplification Mix per reaction | × 25 | μL |
| Total Volume of Amplification Mix to Add to the Diluted | 225 | μL |
| Taq | | |

Add 225 μL of refrigerated Amplification Mix to the microfuge tube containing the 90 μLs of freshly diluted Taq DNA polymerase and vortex briefly to mix. Final volume equals 315 μL. If not amplifying 8 samples, then adjust the appropriate number in the previous equation to determine the required volume.

Pipette into each amplification tube (held on ice):

35 μL Taq and Amplification Mix solution (see step 4)

1-15 μL Genomic DNA (deliver 250 to 500 ng of isolated DNA per tube)

0-14 μL Molecular Biology Grade Water to bring final reaction volume to 50 μL

Cap tubes and temporarily store on ice.

Perform PCR using the following protocol. Transfer the chilled amplification tubes to thermal cycler when it has achieved 94° C.

| | | | |
|---|---|---|---|
| Preheat | | | |
| 94° C.-2 minutes | | | |
| Denature | Anneal | Extend | # Cycles |
| 94° C.-15 seconds | 58° C.-30 seconds | 72° C.-30 | 35 cycles |
| | | seconds | |

Final Extension . . . Hold

| | |
|---|---|
| 72° C.-3 minutes | 4° C. hold. |

This protocol was optimized on a Perkin Elmer Thermal cycler model 9600. It may be necessary to re-optimize these cycling parameters for individual thermalcycling devices and also add mineral oil to the reaction tubes to prevent evaporation in devices that do not have heated lids. The reaction tubes may be removed after the temperature has achieved the 4° C. hold. Store tubes at 4° C. or frozen, if necessary.

### EXAMPLE 3

### ELISA Style Analysis

The amplification products are chemically denatured by alkaline conditions and allowed to bind selectively (hybridize) to sequence-specific or allele-specific oligonucleotide probes in a hybridization solution added to the microwells. The capture probes have been synthesized to contain a terminal base (either 5′ or 3′ terminus of oligonucleotide) modified with a biotin group. The biotin group is a high affinity ligand to streptavidin that is coated onto the microwells. The sequence of the capture olignonucleotide is complementary to the strand of DNA being amplified in a sequence-specific manner by the FITC-modified primer, as described earlier. Thus, the biotin-modified probe will simultaneously bind to the solid support and the denatured (single-stranded) amplification products containing the FITC.

PCR products that are not captured by an allele-specific probe are removed from the microwell by a series of washes. To detect the bound sequences, add an enzyme-conjugated antibody specific for FITC that binds only to the captured sequences. Again, a series of washes removes unbound reporter labels from the microwell. A substrate is added that, when acted upon by the conjugated-enzyme, alkaline phosphatase, turns yellow, indicating a captured sequence and a positive result. Negative results produce substantially less or no color. The microwell can be visually inspected but preferably is read on a microplate reader to quantitate reactivity. One determines the presence of DR52a by comparing the sample absorbance value to an empirically established cutoff value.

### Compositions

Denaturant:

0.3M NaOH: Sigma

0.02% Thymol Blue

Microtiter Plates:

Sreptavidin coated plates: Pierce (Rockford, Ill.) or homemade. Briefly, Streptavidin is diluted into a solution of 10 mM sodium phosphate, 150 mM NaCl, 0.1% Tween 20 (pH 7.1-7.4) to a concentration of 5 ug/mL. 100 uL of this material is added to a microwell and allowed to incubate at 4° C. for 16 hours. Unbound material is removed by several washes with the PBS/Tween buffer described for use during the incubation. A solution of 1% BSA in PBS/Tween is added to block all sites of unreacted plastic in the microwell. After an incubation of 1 hour at 22-27° C., unbound material is again removed by several washes. The plates are ready for use or can be stabilized, dried, and used at a later date.

Hybridization/Probe-containing Buffer:

200 mM Piperazine-N,N′-bis[2-ethanesulfonic acid] (PIPES) Disodium salt (pH 6.8):

Sigma

0.01% Tween 20

580 mM NaCl: Sigma

10 pmol/100 μL Probe: custom synthesis by Genosys (The Woodlands, Tex.)

Probe 1=5′ Biotin modified TTT CGG AGC GGG TGC GG 3′

Wash Buffer 1:

100 mM Tris (pH 7.2)

330 mM NaCl

0.1% Tween 20

Conjugate and buffer:

anti-FITC Fab: Boerhinger Mannheim [Indianapolis, Ind.]

100 mM Tris (pH 7.2)

330 mM NaCl

0.1% Tween 20

0.1% BSA from Boval [Cleburne, Tex.]

Wash Buffer 2:

50 mM Tris (pH7.2)

165 mM NaCl

0.05% Tween 20

Substrate:

para-nitrophenyl phosphate (PNPP) from Zymed [South San Francisco, Calif.]

Substrate Buffer:

10% Diethanolamine (pH 9.7 adjusted with HCl)

0.01% MgCl₂—6H₂O

Stop Solution:

3.0M NaOH

Leave conjugate at 2-8° C. or on ice until needed later. Bring all other reagents to room temperature (22-25° C). During the 4° C. hold in step 6 above, some amplification material may condense on the walls or caps of the reaction tubes. Collect this material into the bottom of the tubes by either centrifuging the tubes or holding the tubes and swinging abruptly and vigorously with wrist or arm.

Remove the caps from each reaction tube, being careful to avoid splashing of amplified material or the formation of aerosols. Pipette 50 μL of Denaturant to each reaction tube, including controls, and allow to stand for five (5) to ten (10) minutes while preparing the microwells. Use a fresh pipette tip for each sample and control. [If mineral oil was used as a vapor barrier during the PCR reaction, then transfer 40-45 μl of the amplified material to a new tube and add an equal volume of the denaturant.]

Remove a dried and stabilized streptavidin-coated microwell strip from a foil pouch and insert into frame. Fill each microwell with 100 μL of the HLA DR52a-Specific Probe. Using a fresh pipette tip for each aliquot, transfer 25 μL of each denatured sample (from above) to a microwell to which has been added the hybridization solution containing the HLA DR52a-Specific Probe. Use a fresh pipette tip for each transfer. Cover microwells with a Plate Sealer and incubate for thirty (30) minutes in a 37° C. dry incubator. Alternatively, a 37° C. water bath may be used in this step and step 19, but be certain not to flood the microwells.

During the incubation, remove 20 mLs of Wash Solution 1. (Store the rest of the reagent at 2-25° C. for later use.) At the same time, transfer 3 mL of the Conjugate Diluent into a fresh 6 mL polypropylene test tube and add 30 μL of Conjugate to the aliquotted Conjugate Diluent. Mix by gently vortexing. After incubation, decant liquids by inverting plate and blotting onto absorbent paper. Add 250 μL of the aliquotted Wash Solution 1 to each well with a multichannel pipettor. Decant liquid and repeat for a total of three (3) washes. Again invert plate and blot on absorbent paper to remove residual fluid. Add 100 μL of diluted Conjugate prepared in step 16 to each microwell. Cover microwells with a Plate Sealer and incubate for about fifteen (15) minutes in 37° C. dry incubator.

During the second incubation, remove 20 mLs of Wash Solution 2. (Store the rest of the reagent at 2-25° C. for later use.) At the same time, prepare the PNPP by dissolving the contents of 1 vial with 0.5 mL of pure water. Store protected from light until use. After incubation, decant liquids by inverting plate and blotting onto absorbent paper. Add 250 μL of prepared Wash Solution 2 to each well with a multichannel pipettor. Decant liquid and repeat for a total of three (3) washes. Again invert plate and blot on absorbent paper to remove residual fluid. Dilute 120 μL of reconstituted PNPP into 3 mL of Substrate Buffer, mix by gently vortexing, then add 100 μL of this solution to each microwell. Cover microwells with a Plate Sealer, and protect from light, then incubate for thirty (30) minutes at room temperature (22-25° C.). To develop an optimal signal, gently shake the plate during this incubation using an orbital shaker.

Stop the reaction by adding 100 μL of Stop Solution to each microwell. Read the absorbance of each well at 405 nm, using a reference wavelength of 650 nm, within 30 minutes of stopping the reaction.

When finished with the assay, incinerate combustible waste materials and decontaminate non-combustible materials with 10% household bleach. Reserve the microwell frame and unused portions of reagents for future use.

The foregoing is considered as illustrative only of the principles of the invention. Furthermore, since numerous modifications and changes will readily occur to those skilled in the art, it is not desired to limit the invention to the exact construction and operation shown and described. Accordingly, all suitable modifications and equivalents fall within the scope of the invention.

We claim:

1. A process for testing genomic DNA for conditions, whether inherited or not inherited, comprising:
a) making a solution comprising the genomic DNA;
b) adding a primer substantially complementary to a diagnostic section of the genomic DNA, selected from the group consisting of a primer having no mismatch bases and a primer having at least one mismatch base;
c) mixing a DNA polymerase into the solution;
d) amplifying the diagnostic section;
e) capturing amplified polynucleotide strands to a solid support; and,
f) detecting captured amplified polynucleotide strands.

2. The process of claim 1 wherein capturing amplified polynucleotide strands comprises hybridizing the strands to a probe.

3. The process of claim 2 further comprising denaturing amplified polynucleotide strands to form single-stranded polynucleotides.

4. The process of claim 3 wherein denaturing comprises separating double-stranded polynucleotides with a process selected from the group consisting of heat denaturing and chemical denaturing.

5. The process of claim 4 wherein denaturing comprises chemical denaturing.

6. The process of claim 5 wherein the probe comprises a polynucleotide for hybridizing to amplified polynucleotide strands.

7. The process of claim 6 wherein the solid support comprises a microtiter plate.

8. The process of claim 7 wherein step g comprises adding a reporter label to the solution.

9. The process of claim 8 wherein the reporter label is selected from the group consisting of enzyme labels, fluorescence labels, luminescent labels, vesicle labels and particle labels.

10. The process of claim 9 wherein the reporter label comprises an enzyme label.

11. The process of claim 7 wherein the microtiter plate comprises a well coated with streptavidin.

12. The process of claim 11 wherein the polynucleotide probe further comprises a biotin compound.

13. A process for detecting a mismatch base in a diagnostic section of genomic DNA for conditions, whether inherited or not inherited, comprising:
a) obtaining the genomic DNA;
b) mixing the genomic DNA with a primer substantially complementary to the diagnostic section of the genomic DNA, selected from the group consisting of a primer having no mismatch bases and a primer having at least one mismatch base;
c) selectively amplifying the diagnostic section from the genomic DNA;
d) capturing amplified polynucleotides to a solid support; and
e) quantifying any complex attached to the solid support.

14. The process of claim 13 further comprising separating amplified polynucleotides of step c into single-stranded polynucleotides.

15. The process of claim 14 wherein separating comprises chemical denaturing.

16. The process of claim 15 further comprising attaching a reporter label to the complex for quantifying presence of the complex.

17. A kit for testing genomic DNA for conditions, whether inherited or not inherited, comprising:
a) a receptacle containing a primer having a nucleotide sequence substantially complementary to a diagnostic section of the DNA;
b) a solid support; and,
c) a receptacle containing a reporter label.

18. The kit of claim 17 further comprising a receptacle containing a probe for attaching to amplified diagnostic sections.

19. The kit of claim 18 further comprising a receptacle containing denaturing compound.

20. The kit of claim 17 wherein a capture probe is attached to the solid support.
